(19) United States
(12) Patent Application Publication
(10) Pub. No.: US 2016/0376329 A1
(43) Pub. Date: Dec. 29, 2016

(54) Glucoprotein for Stimulation of the Immunological System

(71) Applicant: Pedro Brito Correia, Estoril (PT)

(72) Inventor: Pedro Brito Correia, Estoril (PT)

(21) Appl. No.: 14/752,845

(22) Filed: Jun. 27, 2015

(51) Int. Cl. C07K 14/37

(52) U.S. Cl. CPC C07K 14/37

(57) ABSTRACT

Several glucoproteins were isolated from the water extract of mushroom Trametes Versicolor using Fast Protein Liquid Chromatography Gel Filtration.

The immunological efficacy of these glucoproteins were tested in comparison with the extract of the mushroom. We found that the molecule with largest activity against cancer cells was identified by MALDI-TOF and named TVGP1 (Trametes Versicolor Glucoprotein 1).

We claim the molecule TVGP 1 as an important active substance to strengthen the immunological system of humans and animals namely against cancer, some virus and bacteria.

### FIELD OF INVENTION

Immunological system, TLR2 agonist, cancer.

### BACKGROUND OF THE INVENTION

The polysaccharide Krestin (PSK) is extracted from the mushroom Trametes Versicolor, also called Coriolis Versicolor, using water as a solvent. This extract was used since centuries in China and Japan as a treatment for cancer due to its presumed potentiating effects.

Natural extracts have been used in non conventional medicine, with the risks of not knowing the molecules which compose the extract and their possible secondary effects.

In USA and the European Union, the modern standards for approval of the use of a new pharmaceutical product, define the need to have the composition of the active substance, with the intention to protect persons and to test the efficacy and mechanism of action.

Therefore, to obtain the approval for pharmaceutical use, it is necessary to choose the molecule in the natural extract, which is most efficient and to find out if any other molecules in the extract present any synergies.

### DETAILED DESCRIPTION OF THE INVENTION

The PSK extract from Trametes Versicolor was studied by Hailing Lu et al (1), who propose a mechanism for action. PSK is a TLR2 agonist that mediates inhibition of tumor growth by stimulating CD8 T cells and NK cells.

The antigen acts on the toll like receptor, TLR2 on the membrane of the dendritic cell.

This dendritic cell acts on receptor CD8, which is on the membrane of a T-cell.

The T cell produces cytokines , like interleukin-IL2, IL10 and Tumour Necrose Factor TNF.

The cytokines transmit the information to the macrophages cells, which have the ability to transport through their membrane the antigen and metabolize it.

The antigen may be a virus, a bacteria or a cancerous cell.

The mechanism of action of PSK is not limited to the indicated one, but also in a lower scale influences other cells of the immune system.

For the separation of the molecules in the extract, we used a FPLC—Fast Protein Liquid Chromatography, using a chromatograph with a column Superdex 200, diameter 2.6 cm, length 315 cm, with UV detector at 280 nanometer, and range 10-600 kdalton.

The elution liquid contained phosphate at pH=7.4 and sodium chloride 150 mM.

The operation temperature was 5° C.

The results show that the component with 30 kdalton has the highest concentration in the extract.

We claim therefore the use of this gluco protein separately, as the active substance for pharmaceuticals in the increase if immune response namely for cancer treatment.

Determination by MALDI TOF of the gluco protein is underway.

Example of Immune Deficiency Test:

A colorimetric assay based on the MTT method was used to measure the growth inhibition of human colon carcinoma HCT15, HCT116 and SW480 and human endometrium carcinoma RL95 cell lines.

These were placed in microtitre plates of 96 wells containing RPMI 1640 medium supplemented with 100 ml I⁻¹ foetal bovine serum and gentamycin.

Appropriate concentrations of polysaccharides were added to each well (2×10⁵ cells per well) and the cultures were incubated in a CO₂ incubator with 5% CO₂ at 37° C. for 48 h. Cell viability was determined by the addition of 20 micro litres 5 mg/ml MTT to each microtitre well, and after 4 hours of incubation the supernatant was removed and 50 micro litre of dimethyl sulfoxide was added to each well to solubilise the precipitate. The survival rate of human carcinoma cell lines was assayed by measuring the optical density in a microtitre plate reader at 560 nm.

Results were expressed as the inhibition ratio of human carcinoma cell line proliferation as follows

F(%)=100×(A−B)/A

Where A and B are the average numbers of viable tumour cells of the control (i.e. starch) and the test samples respectively. All assays were carried out in triplicate.

We found that the molecule with 30 kdalton has an efficacy 20 times higher than the extract from Trametes Versicolor.

Maldi Tof studies on the molecular structure and Preclinical studies are ongoing.

### INFORMATION DISCLOSURE STATEMENTS

Patents and publications relevant to the patentability of the instant claims, concerning a Glucoprotein for stimulation of the immunological system
1. Hailing Lu et al. Clinical cancer research 17(1) Jan. 1, 2011, Polysaccharide Krestin is a novel TLR2 Agonist that mediates inhibition of tumor growth via stimulation of CD8 T cells and NK cells
2. Yoshihiko Maehara et al. Surgery Today, 2012 January, 42 (1) 8-28, Biological Mechanism and clinical effect of protein-bound polysaccharide K (Krestin): review of development and future perspectives
3. Visnja Popovic et al., Current topics in Medicinal Chemistry, 2013, 13, 000-000, Mycotherapy of cancer: An update on Cytotoxic and antitumor activities of mushrooms, bioactive principles and molecular mechanism of their action
4. Eguchi et al. U.S. Pat. No. 7,790,175, Sep. 7, 2010, Strain of turkey mushrooms, extract from the same and use of the same

1. We claim a molecule isolated from the water extract of the mushroom Trametes Versicolor with a molecular weight of 30 kdalton and a composition consisting of a glucoprotein to be the active substance of a pharmaceutical to be used for reinforcing the human and animal immunological system against oncological cells as well as other antigens.

2. In the product of claim 1 where the process for separation was Fast Protein Liquid Chromatography Gel Filtration.

3. In the product of claim 1 where the previous medical treatment by any other chemical or radiation treatment suitable for cancer, virus or bacteriological disease is followed by administration of the product of claim 1.

4. In the product of claim 1 where the quantity to be administered may vary between 10 and 100 mg per day according to the weight of the person and the reaction to the first administered quantities.
